(19) United States
(12) Patent Application Publication
(10) Pub. No.: US 2025/0134461 A1
(43) Pub. Date: May 1, 2025

(54) Interface for Electrically Coupling a Biosensor to Electrodes of a Wearable Electronic Device

(71) Applicant: Google LLC, Mountain View, CA (US)

(72) Inventors: Seamus David Thomson, Mountain View, CA (US); Lindsey Sunden, San Francisco, CA (US)

(21) Appl. No.: 18/726,053

(22) PCT Filed: Jun. 16, 2022
(86) PCT No.: PCT/US2022/033800

§ 371 Date: Jul. 1, 2024

Related U.S. Application Data
(60) Provisional application No. 63/295,055, filed on Dec. 30, 2021.

(51) Int. Cl. A61B 5/00

(52) U.S. Cl. CPC A61B 5/6826; A61B 5/681; A61B 2562/14

(57) ABSTRACT

An interface for electrically coupling a biosensor to electrodes of a wearable computing device is provided. The interface includes a first portion that includes a first material configured to provide a conductive path between the biosensor and the electrodes of the wearable electronic device. The interface further includes a second portion that is different than the first portion. The second portion includes a second material that is different than the first material and is configured to removably couple the interface to the wearable electronic device such that the first portion contacts the electrodes of the wearable electronic device to provide the conductive path between the biosensor and the electrodes.

### PRIORITY CLAIM

The present application claims priority to U.S. Patent Application No. 63/295,055, titled “Wearable Electronic Device Having a Disposable Interface for a Biosensor,” having a filing date of Dec. 30, 2021, which is incorporated by reference herein.

### FIELD

The present disclosure relates generally to biosensors. More particularly, the present disclosure relates to an interface for electrically coupling a biosensor to electrodes of a wearable electronic device.

### BACKGROUND

A biosensor can be configured to measure a biomarker associated with a body fluid (e.g., blood, sweat). For instance, the biosensor can be placed on a user's skin to measure one or more biomarkers. In some instances, the biosensor can communicate data indicative of the one or more biomarkers to a mobile computing device (e.g., smartphone) configured to process the data. Furthermore, in some instances, the mobile computing device can be configured to display a value for the one or more biomarkers for viewing by the user. For instance, the user can access (e.g., open) a mobile application executing on the mobile computing device to view the value for the one or more biomarkers.

### SUMMARY

Aspects and advantages of embodiments of the present disclosure will be set forth in part in the following description, or can be learned from the description, or can be learned through practice of the embodiments.

In one aspect, an interface for electrically coupling a biosensor to electrodes of a wearable electronic device is provided. The interface includes a first portion and a second portion that is different than the first portion. The first portion includes a first material configured to provide a conductive path between the biosensor and the electrodes of the wearable electronic device. The second portion includes a second material configured to removably couple the interface to the wearable electronic device. Furthermore, the first portion of the interface contacts the electrodes of the wearable electronic device when the interface is removably coupled to the wearable electronic device. In this manner, the first portion of the interface provides the conductive path between the biosensor and the electrodes.

In some embodiments, the second material includes a non-conductive material. For instance, in some embodiments, the non-conductive material includes an adhesive material.

In some embodiments, the second material includes a magnetic material.

In some embodiments, the interface further includes a third portion covering one or more surfaces of the first portion and one or more surfaces of the second portion. Furthermore, the third portion of the interface includes a third material that is different than the first material and different than the second material.

In some embodiments, the second portion of the interface is embedded within the third portion of the interface. In some embodiments, a first surface of the first portion that contacts a first electrode of the electrodes can be flush with a surface of the third portion that contacts the wearable electronic device when the interface is removably coupled to the wearable electronic device. Additionally, a second surface of the first portion that contacts a second electrode of the electrodes can be flush with the surface of the third portion. In alternative embodiments, the first surface of the first portion and the second surface of the first portion can be raised relative to the surface of the third portion that contacts the wearable electronic device when the interface is removably coupled to the wearable electronic device.

In some embodiments, the biosensor can be integrally formed with the third portion of the interface.

In another aspect, a wearable electronic device is provided. The wearable electronic device includes a housing. The wearable electronic device further includes a first electrode and a second electrode. The wearable electronic device even further includes an interface for a biosensor that is separate from the wearable electronic device. The interface is removably coupled to a surface of the housing and includes a first portion and a second portion. The first portion contacts the first electrode and the second electrode and includes a first material configured to provide a conductive path between the biosensor and the electrodes. The second portion is different than the first portion and includes a second material that is different than the first material and configured to removably couple the interface to the surface of the housing.

In some embodiments, the interface further includes a third portion covering one or more surfaces of the first portion and one or more surfaces of the second portion. Furthermore, the third portion of the interface includes a third material that is different than the first material and different than the second material.

In some embodiments, the biosensor can be integrally formed with the third portion of the interface.

In some embodiments, the housing includes a transparent portion and the third portion of the interface includes a transparent region that is positioned over the transparent portion of the housing when the interface is removably coupled to the surface of the housing. In some embodiments, the transparent region of the third portion of the interface includes an aperture defined by the third portion of the interface. In alternative embodiments, the transparent region of the third portion of the interface includes a transparent material.

In some embodiments, the second portion of the interface is embedded within the third portion of the interface. In some embodiments, a first surface of the first portion that contacts the first electrode is raised relative to a surface of the third portion that contacts the surface of the housing when the interface is removably coupled to the surface of the housing. Additionally, a second surface of the first portion that contacts the second electrode is raised relative to the surface of the third portion. Still further, in such embodiments, a surface of the first electrode is raised relative to the surface of the housing and a surface of the second electrode is raised relative to the surface of the housing.

In some embodiments, the housing has a ring shape to accommodate a finger of a user wearing the wearable electronic device.

In some embodiments, the second material includes at least one of an adhesive material or a magnetic material.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 depicts a wearable electronic device according to an embodiment of the present disclosure.

FIG. 2 depicts a rear perspective view of a wearable electronic device according to an embodiment of the present disclosure.

FIG. 3 depicts a cross-sectional view of an interface for electrically coupling a biosensor to electrodes of a wearable electronic device according to an embodiment of the present disclosure.

FIG. 4 depicts a bottom view of the interface of FIG. 3 according to an embodiment of the present disclosure.

FIG. 5 depicts the interface of FIG. 3 removably coupled to the wearable electronic device of FIG. 2 according to an embodiment of the present disclosure.

FIG. 6 depicts a rear perspective view of a wearable electronic device according to an embodiment of the present disclosure.

FIG. 7 depicts a bottom view of an interface for electrically coupling a biosensor to electrodes according to an embodiment of the present disclosure.

FIG. 8 depicts the interface of FIG. 7 removably coupled to the wearable electronic device of FIG. 6 according to an embodiment of the present disclosure.

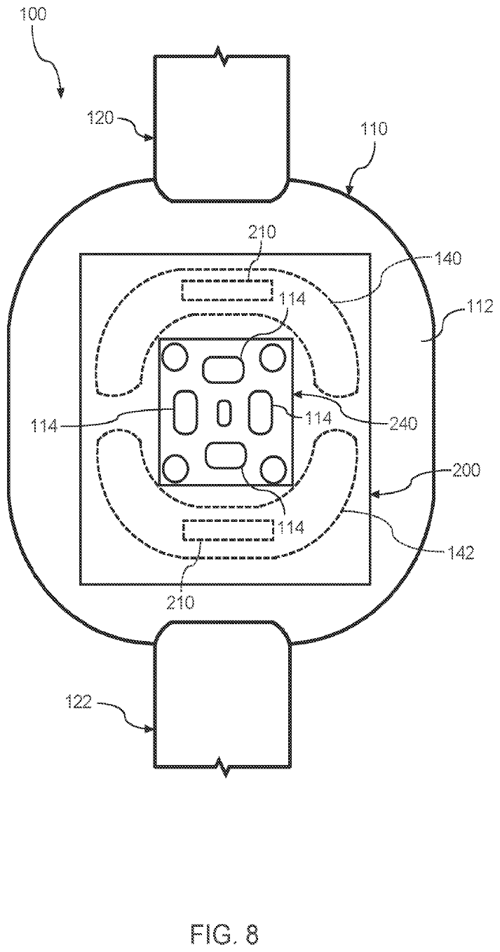

FIG. 9 depicts an interface for electrically coupling a biosensor to electrodes of wearable electronic device removably coupled to the wearable electronic device according to an embodiment of the present disclosure.

FIG. 10 depicts a cross-sectional view of an interface for electrically coupling a biosensor to electrodes of a wearable electronic device according to an embodiment of the present disclosure.

FIG. 11 depicts a bottom view of the interface of FIG. 10 according to an embodiment of the present disclosure.

FIG. 12 depicts a cross-sectional view of an interface for electrically coupling a biosensor to electrodes of a wearable electronic device according to an embodiment of the present disclosure.

FIG. 13 depicts the interface of FIG. 12 aligned with electrodes of a wearable electronic device according to an embodiment of the present disclosure.

FIG. 14 depicts a cross-sectional view of an interface for electrically coupling a biosensor to electrodes of a wearable electronic device according to an embodiment of the present disclosure.

FIG. 15 depicts a bottom view of the interface of FIG. 14 according to an embodiment of the present disclosure.

FIG. 16 depicts a system for obtaining biosensor data according to an embodiment of the present disclosure.

### DETAILED DESCRIPTION

Reference now will be made in detail to embodiments of the present disclosure, one or more examples of which are illustrated in the drawings. Each example is provided by way of explanation of the present disclosure, not limitation of the present disclosure. In fact, it will be apparent to those skilled in the art that various modifications and variations can be made in the present disclosure without departing from the scope or spirit of the disclosure. For instance, features illustrated or described as part of one embodiment can be used with another embodiment to yield a still further embodiment. Thus, it is intended that the present invention covers such modifications and variations as come within the scope of the appended claims and their equivalents.

The present disclosure is directed to biosensors that adhere to or are in close contact with a user's skin. Biosensors can be configured to measure biomarkers associated with a body fluid (e.g., blood, sweat, etc.) of the user. Biosensors can also implement electrochemical measurements. However, biosensors require electronics in order to perform electrical sensing methods that include electrochemical techniques (e.g., cyclic voltammetry, chronopotentiometry) associated with the electrochemical measurements. Furthermore, the electronics generally need to have a direct electrical connection to the biosensor. The electronics can be separate from the biosensor or, alternatively, can be integral with the biosensor. However, in either case, the electronics are sizable (e.g., bulky) and have limited reusability.

Example aspects of the present disclosure are directed to an interface (e.g., a patch) for electrically coupling a biosensor to electrodes of a wearable electronic device. For instance, the wearable electronic device can be worn on a user's wrist and the user can contact (e.g., touch) the electrodes that the user can contact (e.g., touch) to obtain a biometric measurement (e.g., electrocardiogram, electrodermal activity). It should be understood that the wearable electronic device can include one or more processors and one or more memory devices configured to store computer-readable instructions that, when executed by the one or more processors, cause the one or more processors to perform operations. For example, in some embodiments, the operations can include processing electrical signals indicative of the user touching the electrodes to perform the biometric measurement. Details of the interface will now be discussed in detail.

The interface includes a first portion that can be positioned on the electrodes of the wearable electronic device. The first portion of the interface can include a conductive material (e.g., copper). In this manner, the first portion of the interface can provide a conductive path from the electrodes to a biosensor. More particularly, a first region of the conductive path can extend between the biosensor and a first electrode of the wearable electronic device and a second region of the conductive path can extend between the biosensor and a second electrode of the wearable electronic device. In some embodiments, a driving or excitation signal can be provided to the biosensor via the conductive path to facilitate electrochemical techniques that are associated with electrochemical measurements performed to obtain biometric information about the user.

The interface further includes a second portion that is different than the first portion. In some embodiments, the interface can be positioned entirely on a portion of the wearable electronic device that does not include the electrodes. In alternative embodiments, the second portion of the interface can be partially positioned on the electrodes.

The second portion of the interface can include a second material that is different than the first material. For instance, in some embodiments, the second material can include a non-conductive material. The non-conductive material can, in some embodiments, include an adhesive material. In this manner, the interface can be secured to the wearable electronic device via the adhesive material. For instance, the interface can adhere to a surface of the wearable electronic device via the adhesive material. In alternative embodiments, the second portion of the interface can include a magnetic material. In such embodiments, the interface (e.g., the second portion thereof) can be magnetically coupled to the wearable electronic device (e.g., a housing thereof).

Since the second portion of the interface includes a second material operable to removably couple the interface to the wearable electronic device (e.g., a housing there of), it should be understood that the interface is disposable. As used herein, the term “disposable” means a first instance of the interface that is electrically coupling the biosensor to the electrodes of the wearable electronic device can be replaced with a second instance of the interface that can electrically couple the biosensor to the electrodes of the wearable electronic device or, alternatively, can electrically couple a different biosensor to the electrodes of the wearable electronic device.

The interface can include a third portion that is different than the first portion and the second portion. For instance, the third portion of the interface can include a third material that is different than the first material and the second material. In some embodiments, the third portion of the interface can cover surfaces of the first and second portions that do not contact the wearable electronic device. In this manner, the third portion of the interface can provide a cover for the first and second portions of the interface.

In some embodiments, the portions (e.g., first portion, second portion, third portion) of the interface can have different thicknesses. For example, the third portion of the interface can be thicker than the first portion of the interface and the second portion of the interface. Furthermore, in some embodiments, the first portion of the interface can be thicker than the second portion of the interface.

In some embodiments, the wearable electronic device can include an optical sensor (e.g., photoplethysmography (PPG) sensor) disposed with a housing of the wearable electronic device. The optical sensor can be configured to emit a light signal (e.g., infrared light) that passes through a transparent portion of the housing. Furthermore, the light signal penetrates the user's skin and blood vessels and returns to the optical sensor as a reflected light signal. It should be understood, using the disclosures provided herein, that one or more biometrics associated with the user can be determined based, at least in part, on the reflected light signal.

In some embodiments, the interface can define an aperture (e.g., opening) extending therethrough and surrounding the transparent portion of the housing through which the light signal and the reflected light signal travel. In this manner, the interface can avoid interfering (e.g. blocking) with an optical path defined between the optical sensor and the user. In alternative embodiments, the interface can include a transparent material. Furthermore, the transparent material can be positioned along the optical path when the interface is coupled to the wearable electronic device. In this manner, the interface can avoid interfering with the optical path.

In some embodiments, the biosensor can be integral (e.g., part of) with the interface. For instance, the biosensor can be included within one of the portions (e.g., third portion) of the interface. In alternative embodiments, the biosensor can be separate from the interface. For instance, the biosensor can include an electrical connection to facilitate electrically coupling the biosensor to the first portion of the interface.

In some embodiments, the interface can include a microfluidic system. For instance, the microfluidic system can include a plurality of channels implemented in one or more portions (e.g., second portion, third portion) of the interface. It should be understood, however, that the interface can include any suitable structure to facilitate collection of a body fluid (e.g., blood, sweat, etc.) on which measurements can be performed to determine one or more biomarkers for a user.

An interface according to example aspects of the present disclosure can provide numerous technical effects and benefits. For instance, the interface can provide an electrical connection (e.g., conductive path) between the biosensor and the electrodes of the wearable electronic device. Furthermore, since the biosensor can be electrically coupled to the electrodes of the wearable electronic device and therefore leverage the one or more processors of the wearable electronic device, a footprint of the biosensor can be reduced since the biosensor no longer needs its own electronics to facilitate electrochemical techniques.

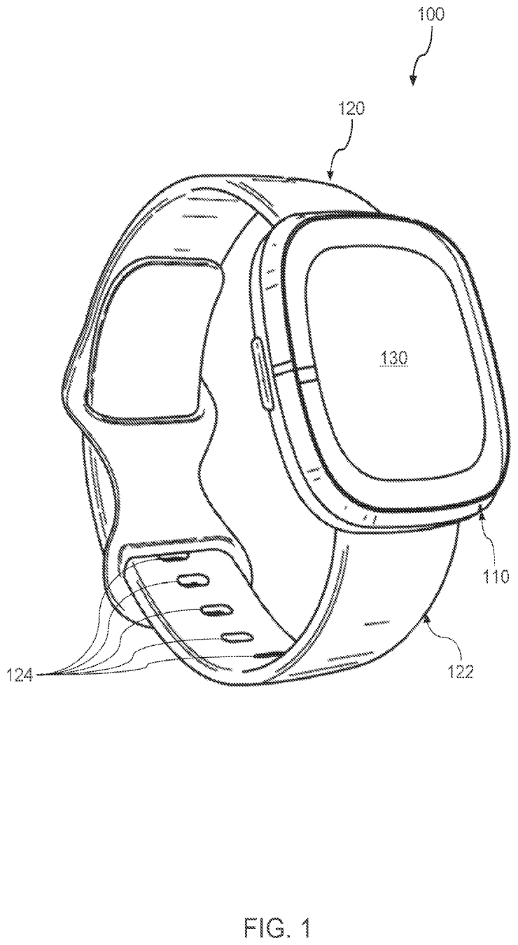

Referring now to the FIGS., FIG. 1 depicts a wearable electronic device 100 according to some embodiments of the present disclosure. The wearable electronic device 100 can be worn, for instance, on an arm (e.g., wrist) of a user. The wearable electronic device 100 can include a housing 110 and one or more electronic components (e.g., disposed on printed circuit boards) disposed within an internal cavity defined by the housing 110. Furthermore, the wearable electronic device 100 can include a battery (not shown) that is disposed within the internal cavity of the housing 110.

In some embodiments, the wearable electronic device 100 can include a first band 120 coupled to the housing 110 at a first location and a second band 122 coupled to the housing 110 at a second location. The first band 120 and the second band 122 can be coupled to one another to secure the housing 110 to the arm of the user. For instance, the first band 120 can include a buckle or clasp (not shown). Additionally, the second band 122 can define a plurality of apertures 124 spaced apart from one another along a length of the second band 122. In such embodiments, a prong of the buckle associated with the first band 120 can extend through one of the plurality of openings defined by the second band 132 to couple the first band 120 to the second band 122.

It should be appreciated that the first band 120 can be coupled to the second band 122 using any suitable type of fastener. For instance, in some embodiments, the first band 120 and the second band 122 can include a magnet. In such embodiments, the first band 120 and the second band 122 can be magnetically coupled to one another to secure the housing 110 to the arm of the user.

In some embodiments, the wearable electronic device 100 can include a display 130 configured to display content (e.g., time, date, biometric, notifications, etc.) for viewing by the user. For instance, the display 130 can include a plurality of pixels. In some embodiments, the display 130 can include an organic light emitting diode (OLED) display. It should be understood, however, that the display 130 can include any suitable type of display.

The wearable electronic device 100 can include a first electrode 140 and a second electrode 142. It should be understood that the wearable electronic device 100 can include more or fewer electrodes. As shown, the first electrode 140 and the second electrode 142 can, in some embodiments, be positioned on a lower portion (e.g., wrist-facing side) of the housing 110. More specifically, the electrodes 140, 142 can be positioned within respective apertures (e.g., cutouts) defined by the lower portion of the housing 110. In this manner, the first electrode 140 and the second electrode 142 can each contact (e.g., touch) the user's skin (e.g., wrist) when the user is wearing the wearable electronic device 100. Furthermore, since the electrodes 140, 142 contact the user's skin, the electrodes 140, 142 can be used to measure one or more biometrics (e.g., electrodermal activity, electrocardiogram) of the user.

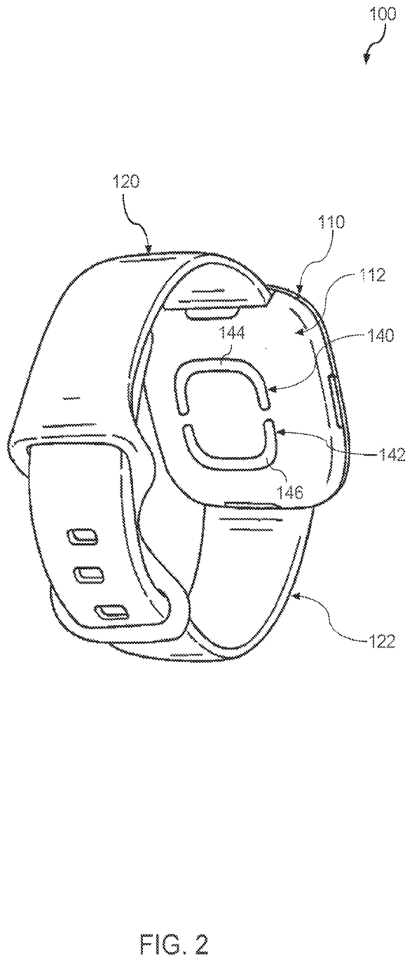

Although the wearable electronic device 100 of FIGS. 1 and 2 is depicted as a wrist-watch, it should be understood that the wearable electronic device 100 is not intended to be limited in this manner. In some embodiments, the wearable electronic device 100 can be a ring that can be worn on the user's finger. In alternative embodiments, the wearable electronic device 100 can be configured as a hearing device (e.g., earbud) that can be inserted into an ear canal of a user's ear. It should be understood that other embodiments of the wearable electronic device 100 (e.g., ring, hearing device) can include electrodes that are the same as or similar to the electrodes (e.g., first electrode 140, second electrode 142) discussed above with reference to FIG. 2.

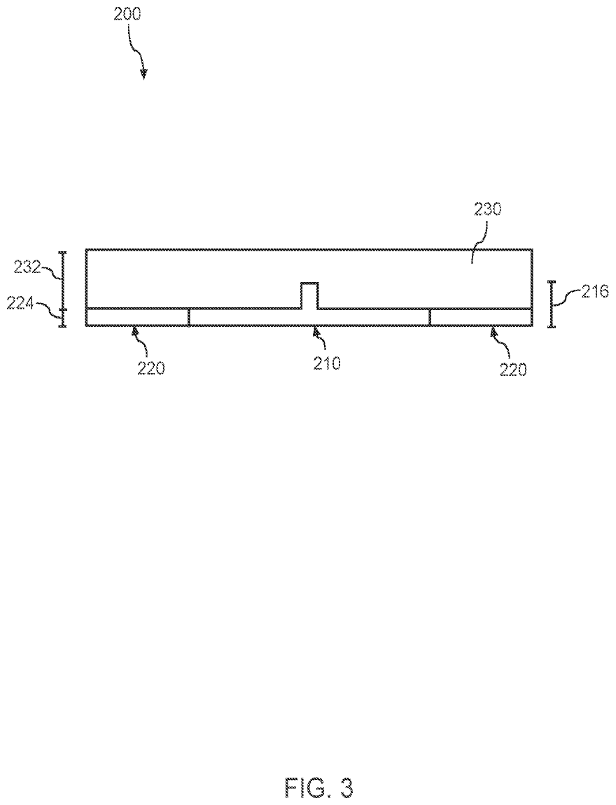

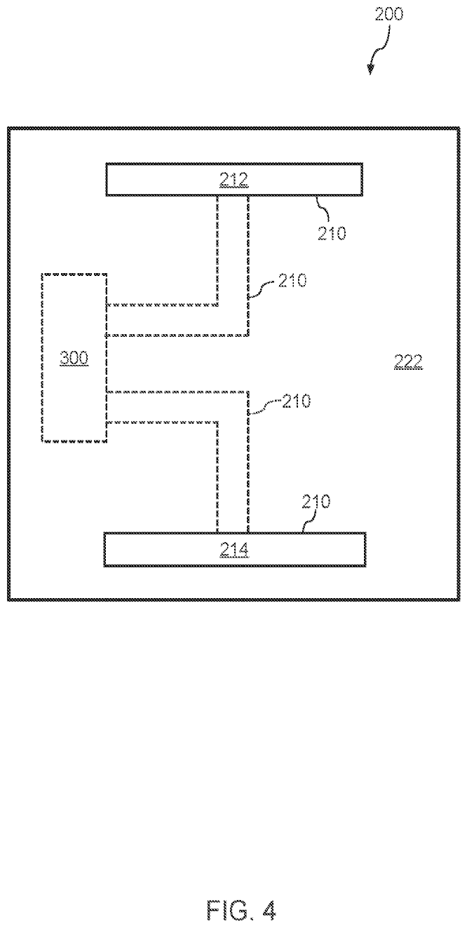

Referring now to FIGS. 3 and 4, an interface 200 for electrically coupling a biosensor 300 to electrodes 140, 142 (FIG. 1) of the wearable electronic device 100 (FIG. 1) is provided according to an embodiment of the present disclosure. As shown, the interface 200 includes a first portion 210, a second portion 220, and a third portion 230. Details of each of the portions 210, 220, 230 will now be discussed.

The first portion 210 of the interface 200 contacts (e.g., touches) the electrodes 140, 142 (FIG. 2) of the wearable electronic device 100 (FIG. 1) when the interface 200 is removably coupled to the wearable electronic device 100. For instance, in some embodiments, the first portion 210 of the interface 200 can include a first surface 212 that contacts electrode 140 (FIG. 2) and a second surface 214 that is different than the first surface 212 and contacts electrode 142 (FIG. 2). In this manner, the first portion 210 of the interface 200 provides a conductive path between the biosensor 300 and electrodes 140, 142. More specifically, the conductive path includes a first region extending from electrode 140 to the biosensor 300 and a second region extending from electrode 142 to the biosensor 300.

The first portion 210 of the interface 200 includes a first material, such as a conductive material (e.g., copper, a conductive polymer such as poly (3,4-ethylenedioxythiophene) polystyrene sulfonate (PEDOT:PSS)). In this manner, the first portion 210 of the interface 200 can provide a conductive path between the biosensor 300 and the electrodes 140, 142 of the wearable electronic device 100.

The second portion 220 of the interface 200 includes a second material that is different than the first material. It should be understood that the second material can include any material or combination of materials that allow the interface 200 to be removably coupled to the wearable electronic device 100. For instance, the second material can include an adhesive material, a magnetic material, or both.

In some embodiments, a first surface 222 of the second portion 220 of the interface 200 contacts the wearable electronic device 100. For instance, in some embodiments, the entirety of the first surface 222 of the second portion 220 contacts an exterior surface 112 (FIG. 2) of the housing 110 (FIG. 2) of the wearable electronic device 100. In alternative embodiments, a first portion of the first surface 222 contacts electrode 140, a second portion of the first surface 222 contacts electrode 142, and a third portion of the first surface 222 contacts the exterior surface 112 (FIG. 2) of the housing 110.

In some embodiments, a surface area of the second portion 220 of the interface 200 can be different than a surface area of the first portion 210 of the interface 200. For instance, the surface area of the second portion 220 of the interface 200 can be greater (e.g., larger) than the surface area of the first portion 210 of the interface 200. In alternative embodiments, the surface area of the second portion 220 of the interface 200 can be the same (e.g., equal to) or smaller than the surface area of the first portion 210 of the interface 200.

The third portion 230 of the interface 200 can cover one or more surfaces of the first and second portions 210, 220. For instance, in some embodiments, the third portion 230 of the interface 200 can cover one or more surfaces of the first and second portions 210, 220 that do not contact (e.g., touch) the wearable electronic device 100 (FIG. 1) when the interface 200 is removably coupled to the wearable electronic device 100.

In some embodiments, the third portion 230 of the interface 200 includes one or materials that provide structural integrity and/or flexibility to the interface 200. Examples of the one or more materials can include, without limitation, synthetic polymers such as poly (ethylene terephthalate) (PET) or siloxane elastomers such as poly (dimethylsiloxane) (PDMS). The latter material (that is, siloxane elastomers) is both flexible, optically transparent, and commonly used for creating microfluidic channels in which biosensors (e.g., biosensor 300) can reside.

It should be understood, however, that the third portion 230 of the interface 200 can include any suitable material. It should also be understood that connection and/or bonding between first, second, and third portions 210, 220, 230 can be mechanical or chemical in nature. For chemical bonding, several techniques can be applied to enable this with examples such as use of chemical adhesives or bonding via plasma surface treatments.

In some embodiments, the first portion 210 of the interface 200, the second portion 220 of the interface 200, and the third portion 230 of the interface 200 can each have a different thickness. For instance, the first portion 210 of the interface 200 can have a first thickness 216, the second portion 220 of the interface 200 can have a second thickness 224 that is different than the first thickness 216, and the third portion 230 of the interface 200 can have a third thickness 232 that is different than the first thickness 216 and the second thickness 224.

In alternative embodiments, two or more of the portions 210, 220, 230 of the interface 200 can have the same thickness. For instance, the first thickness 216 of the first portion 210 can be the same as (e.g., equal to) the second thickness 224 of the second portion 220.

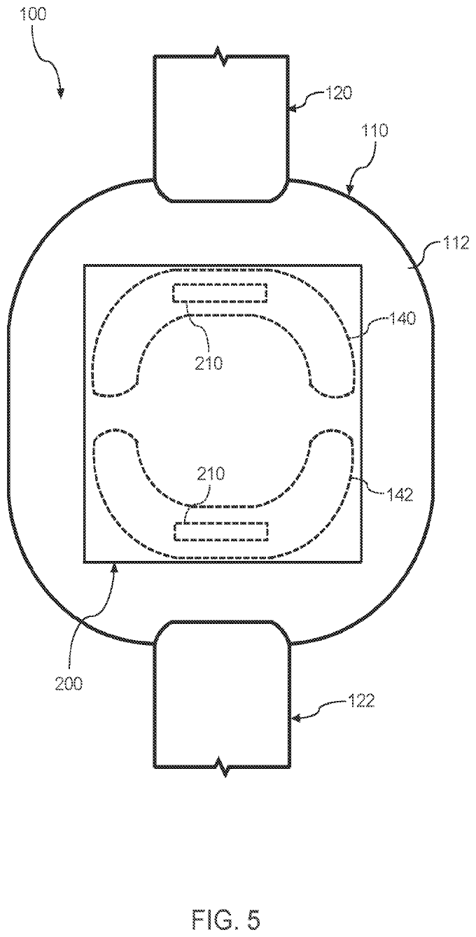

Referring now to FIG. 5, the interface 200 is removably coupled to the wearable electronic device 100 according to an embodiment of the present disclosure. As shown, the interface 200 can be removably coupled to the housing 110 of the wearable electronic device 100. For instance, in some embodiments, at least a portion of the first surface 222 (FIG. 4) of the second portion 220 (FIG. 4) of the interface 200 can contact (e.g., touch) an exterior surface 112 of the housing 110. Furthermore, the first surface 212 (FIG. 4) of the first portion 210 (denoted by dashed lines) of the interface 200 can contact electrode 140 of the wearable electronic device 100 and the second surface 214 (FIG. 4) of the first portion 210 of the interface 200 can contact electrode 142 of the wearable electronic device 100. In this manner, the first portion 210 of the interface 200 can provide a conductive path between electrodes 140, 142 and the biosensor 300 (FIG. 4) that is separate from the wearable electronic device 100.

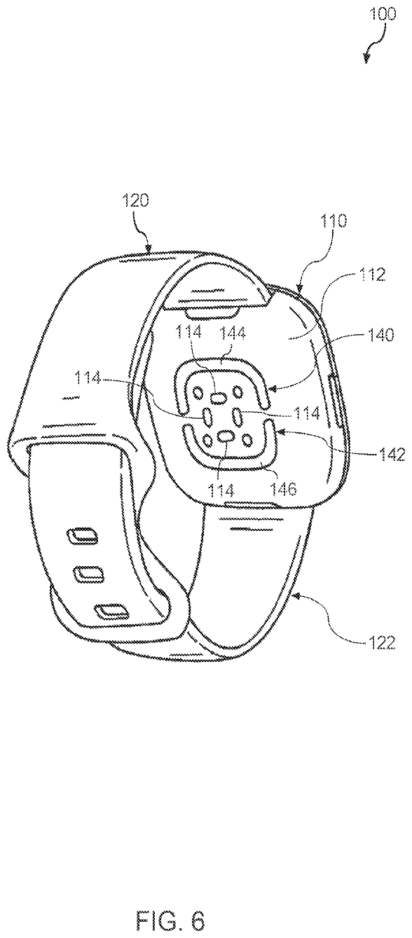

Referring now to FIG. 6, the wearable electronic device 100 can, in some embodiments, include one or more optical sensors (e.g., photoplethysmography (PPG) sensors) disposed within an interior cavity defined by the housing 110. The optical sensor(s) can be configured to emit a light signal (e.g., infrared light) that passes through a transparent portion 114 of the housing 110. For instance, in some embodiments, the transparent portion 114 of the housing 110 can include one or more windows that are transparent and positioned at different locations (e.g., cutouts) on the housing 110. It should be understood that the light signal exits the interior cavity of the housing 110 via the transparent portion 114 thereof, penetrates the user's skin and blood vessels, and returns to the optical sensor(s) as a reflected light signal. It should be understood, using the disclosures provided herein, that one or more biometrics associated with the user can be determined based, at least in part, on the reflected light signal.

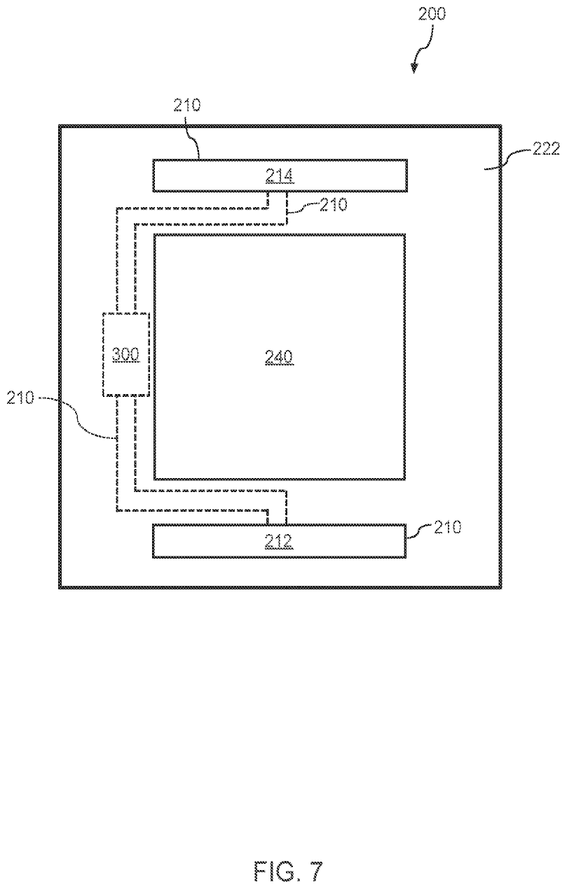

Referring now to FIG. 7, the interface 200 can, in some embodiments, include a transparent region 240 to accommodate the transparent portion 114 (FIG. 6) of the housing 110 (FIG. 6). More particularly, the transparent region 240 of the interface 200 can be positioned over the transparent portion 114 (FIG. 6) of the housing 110 (FIG. 6) of the wearable electronic device 100 (FIG. 6) when the interface 200 is removably coupled to the exterior surface 112 (FIG. 6) of the housing 110. In this manner, the light signal emitted from the optical sensor(s) and exiting the internal cavity of the housing 110 through the transparent portion 114 thereof can then pass through the transparent region 240 of the interface 200. Furthermore, the reflected light signal can pass through the transparent region 240 of the interface 200 and enter the internal cavity of the housing 110 via the transparent portion 114 of the housing 110.

In some embodiments, the transparent region 240 of the interface 200 can include an aperture defined by the interface 200. For instance, the aperture can be defined by the third portion 230 (FIG. 3) of the interface 200. It should be understood that the aperture extends through the third portion 230 of the interface 200. It should also be understood that the aperture is large enough so that the interface 200 does not obstruct (e.g., cover) the transparent portion 114 of the housing 110 when the interface 200 is removably coupled to the exterior surface 112 of the housing 110.

In alternative embodiments, the transparent region 240 can correspond to a region of the third portion 230 (FIG. 3) of the interface 200. Furthermore, the transparent region 240 can include a transparent material and can be positioned over the transparent portion 114 of the wearable electronic device 100 when the interface 200 is removably coupled to the housing 110 of the wearable electronic device 100. In this manner, the light signal emitted from the optical sensor(s) and exiting the internal cavity of the housing 110 through the transparent portion 114 of the housing 110 can pass through the transparent region 240 of the third portion 230 of the interface 200. Furthermore, reflected light signals can pass through the transparent region 240 of the third portion 230 of the interface 200 and enter the internal cavity of the housing 110 via the transparent portion 114 of the housing 110.

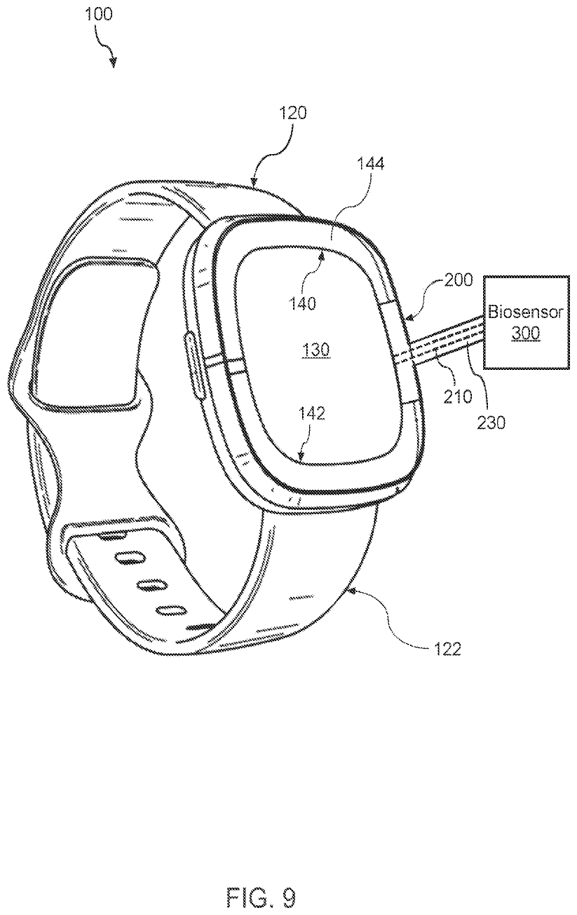

Referring now to FIG. 9, the electrodes 140, 142 of the wearable electronic device 100 can, in some embodiments, be positioned on an upper portion (e.g., non-wrist facing side) of the housing 110. It should be understood that the upper portion of the wearable electronic device 100 can include any portion of the wearable electronic device 100 that does not face and/or contact a user's wrist when the wearable electronic device 100 is worn by the user. In some embodiments, the housing 110 can include a transparent cover that is positioned over the display 130 to protect the display 130 from being damaged (e.g., scratched, cracked). In some embodiments, the electrodes 140, 142 can be positioned on the transparent cover. For instance, the electrodes 140, 142 can be positioned on a region of the transparent cover that corresponds to a dead-band region of the display 130. In this manner, the electrodes 140, 142 can eliminate the need for a decorative element (e.g., decorative bezel) to cover the dead-band region of the display 130.

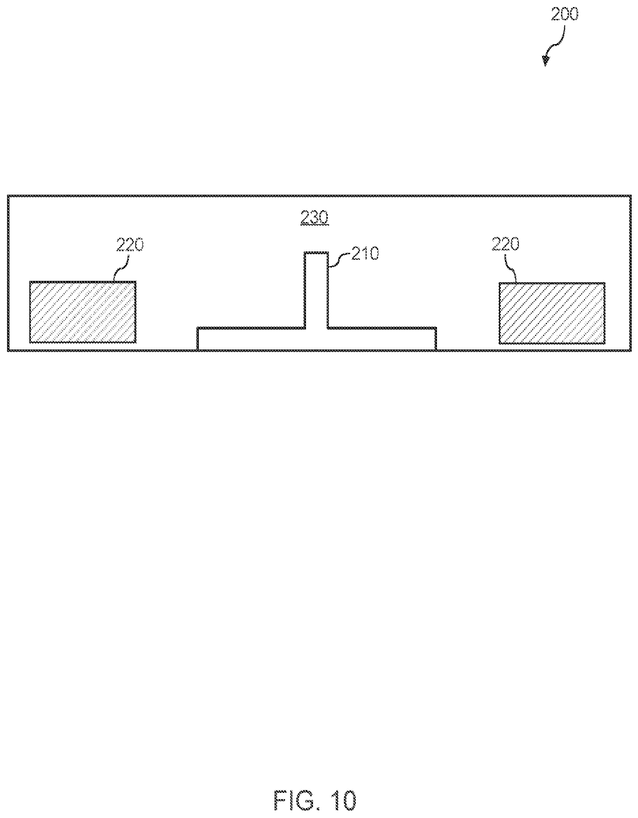

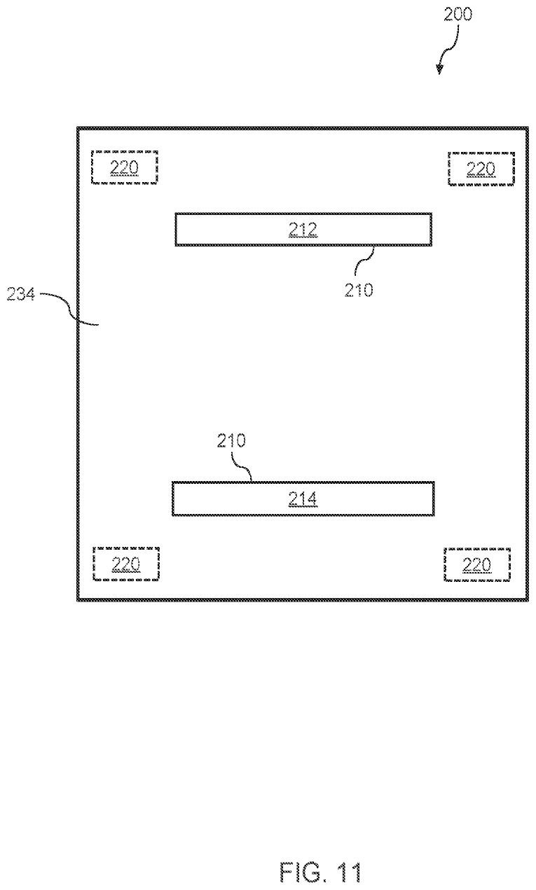

Referring now to FIGS. 10 and 11, a cross-sectional view (FIG. 10) and a bottom view (FIG. 11) of the interface 200 is provided according to an embodiment of the present disclosure. As shown, the second portion 220 of the interface 200 can be embedded within the third portion 230 of the interface 200. Furthermore, the first and second surfaces 212, 214 of the first portion 210 of the interface 200 can be flush with a first surface 234 of the third portion 230 of the interface.

It should be understood that the first surface 234 of the third portion 230 of the interface 200 contacts a surface of the wearable electronic device 100 when the interface 200 is magnetically coupled to the wearable electronic device 100. For instance, in some embodiments, the first surface 234 of the third portion 230 of the interface 200 contacts the exterior surface 112 (FIG. 2) of the wearable electronic device 100.

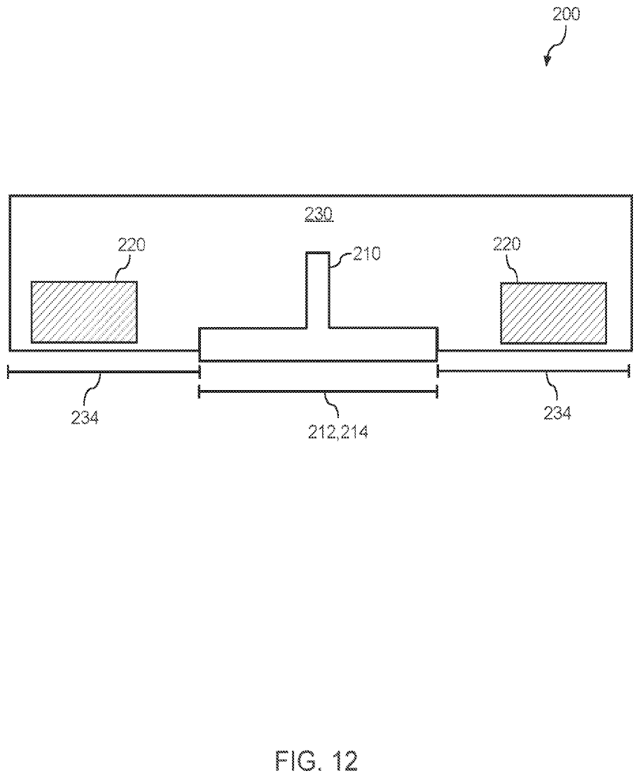

Referring now to FIG. 12, a cross-sectional view of the interface 200 is provided according to another embodiment of the present disclosure. The interface 200 depicted in FIG. 12 can be similar to the interface 200 discussed above with reference to FIGS. 10 and 11. For instance, the second portion 220 of the interface 200 in FIG. 12 can be embedded within the third portion 230 of the interface 200 and can include a magnetic material to promote magnetically coupling the interface 200 to the wearable electronic device 100. However, in contrast to the interface 200 of FIGS. 10 and 11, the first portion 210 of the interface 200 in FIG. 12 can be raised relative to the first surface 234 of the third portion 230 of the interface 200.

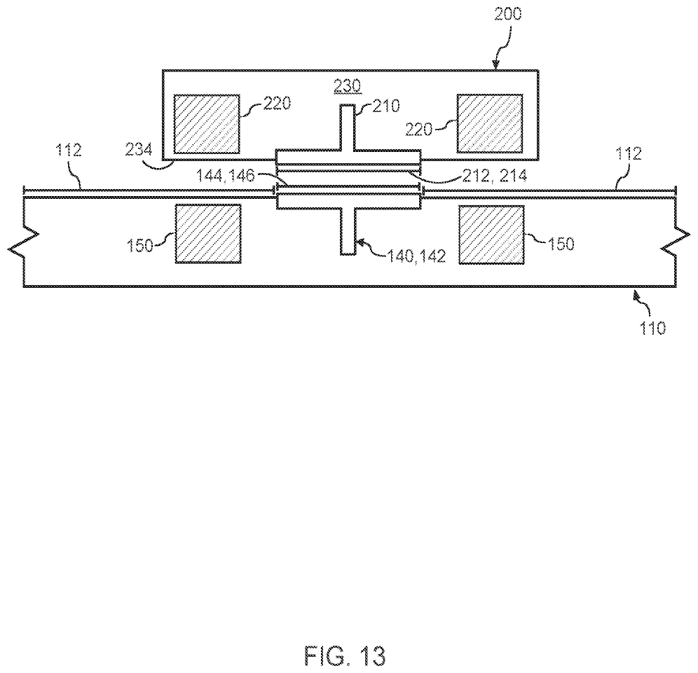

Referring now to FIG. 13, surface 144, 146 of electrodes 140 and 142, respectively, can be raised relative to the exterior surface 112 of the housing 110 of the wearable electronic device 100. In this manner, the first portion 210 of the interface 200, specifically the first surface 212 and second surface 214 thereof, can make better contact with the electrodes 140, 142 of the wearable electronic device 100, specifically surface 144 of electrode 140 and surface 146 of electrode 142.

Furthermore, the wearable electronic device 100 can include magnetic material 150 to facilitate magnetically coupling the interface 200 to the wearable electronic device 100. For instance, in some embodiments, the magnetic material 150 can include a plurality of magnets disposed within the internal cavity defined by the housing 110 of the wearable electronic device 100. In alternative embodiments, a portion of the exterior surface 112 of the housing 110 can include the magnetic material 150.

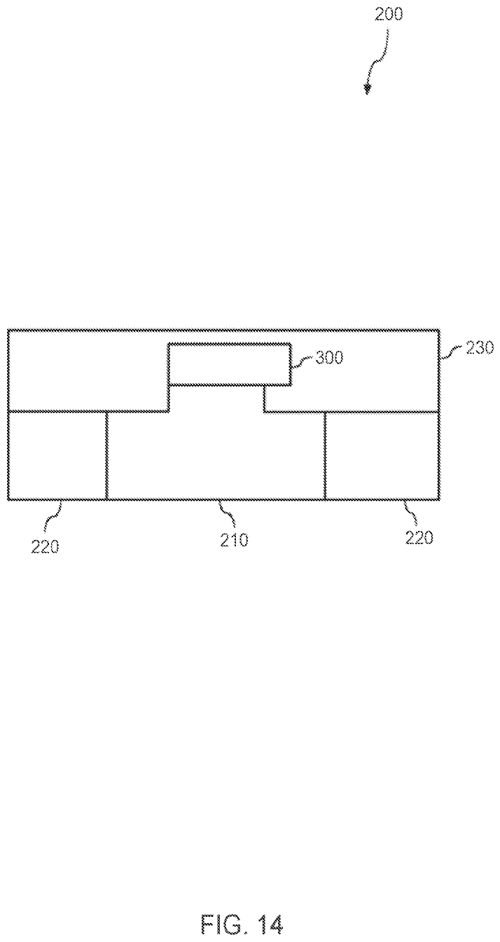

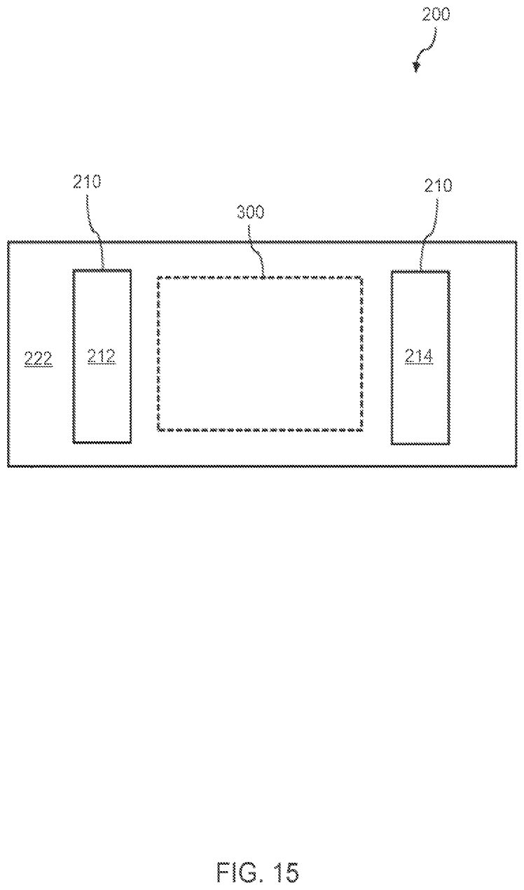

Referring now to FIGS. 14 and 15, a cross-sectional view (FIG. 14) and a bottom view (FIG. 15) of the interface 200 are provided according to an embodiment of the present disclosure. As shown, the interface 200 can include the biosensor 300. For instance, in some embodiments, the biosensor 300 can be integrally formed with the third portion 230 of the interface 200. Furthermore, in some embodiments, the interface 200 can include a microfluidic system that includes a plurality of channels. In such embodiments, the plurality of channels can be defined by the third portion 230 of the interface 200.

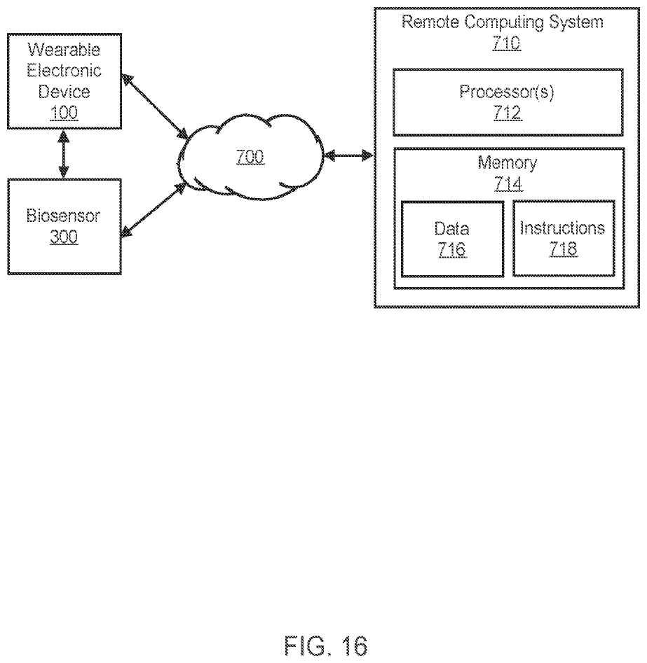

Referring now to FIG. 16, the wearable electronic device 100 and the biosensor 300 can be communicatively coupled to a network 700. The biosensor 300 can communicate data (e.g., biometric measurements) over the network 700 to a remote computing system 710 (e.g., internet-of-things device) for storage and/or processing. For instance, in some embodiments, the biosensor 300 can communicate the data to wearable electronic device 100. In such embodiments, the biosensor 300 can communicate the data to the wearable electronic device 100 and then the wearable electronic device 100 can communicate the data over the network 700 to the remote computing system 710. In alternative embodiments, biosensor 300 can bypass the wearable electronic device 100 and instead communicate the data directly to the remote computing system 710 via the network 700.

The remote computing system 710 includes one or more processors 712 and a memory 714. The one or more processors 712 can be any suitable processing device (e.g., a processor core, a microprocessor, an ASIC, an FPGA, a controller, a microcontroller, etc.) and can be one processor or a plurality of processors that are operatively connected. The memory 714 can include one or more non-transitory computer-readable storage media, such as RAM, ROM, EEPROM, EPROM, flash memory devices, magnetic disks, etc., and combinations thereof. The memory 714 can store data 716 and instructions 718 which are executed by the processor 712 to cause the remote computing system 710 to perform operations, such as any of the operations described herein.

In some embodiments, the remote computing system 710 includes or is otherwise implemented by one or more computing devices. In instances in which the remote computing system 710 includes plural server computing devices, such server computing devices can operate according to sequential computing architectures, parallel computing architectures, or some combination thereof.

The network 700 can be any type of communications network, such as a local area network (e.g., intranet), wide area network (e.g., Internet), or some combination thereof and can include any number of wired or wireless links. In general, communication over the network 700 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. An interface for electrically coupling a biosensor to electrodes of a wearable electronic device, the interface comprising:
a first portion that includes a first material configured to provide a conductive path between the biosensor and the electrodes of the wearable electronic device;
a second portion that is different than the first portion, the second portion including a second material that is different than the first material, the second material configured to removably couple the interface to the wearable electronic device,
wherein when the interface is removably coupled to the wearable electronic device, the first portion of the interface contacts the electrodes of the wearable electronic device to provide the conductive path between the biosensor and the electrodes.

2. The interface of claim 1, wherein the second material includes a non-conductive material.

3. The interface of claim 2, wherein the non-conductive material includes an adhesive material.

4. The interface of claim 1, wherein the second material includes a magnetic material.

5. The interface of claim 1, further comprising:
a third portion covering one or more surfaces of the first portion and one or more surfaces of the second portion, the third portion including a third material that is different than the first material and the second material.

6. The interface of claim 5, wherein the second portion is embedded within the third portion.

7. The interface of claim 6, wherein:
a first surface of the first portion that contacts a first electrode of the electrodes is flush with a surface of the third portion that contacts the wearable electronic device when the interface is removably coupled to the wearable electronic device; and
a second surface of the first portion that contacts a second electrode of the electrodes is flush with the surface of the third portion.

8. The interface of claim 6, wherein:
a first surface of the first portion that contacts a first electrode of the electrodes is raised relative to a surface of the third portion that contacts the wearable electronic device when the interface is removably coupled to the wearable electronic device; and
a second surface of the first portion that contacts a second electrode of the electrodes is raised relative to the surface of the third portion.

9. The interface of claim 5, wherein the biosensor is integrally formed with the third portion of the interface.

10. A wearable electronic device comprising:
a housing;
a first electrode and a second electrode; and
an interface for electrically coupling a biosensor to the first electrode and the second electrode, the interface including:
a first portion contacting the first electrode and the second electrode, the first portion including a first material configured to provide a conductive path between the biosensor and the first and second electrodes; and
a second portion that is different than the first portion, the second portion including a second material that is different than the first material and operable to removably couple the interface to a surface of the housing.

11. The wearable electronic device of claim 10, wherein the interface further includes:
a third portion covering one or more surfaces of the first portion and one or more surfaces of the second portion, the third portion including a third material that is different than the first material and the second material.

12. The wearable electronic device of claim 11, wherein the biosensor is integrally formed with the third portion of the interface.

13. The wearable electronic device of claim 11, wherein:
the housing includes a transparent portion; and
the third portion of the interface includes a transparent region that is positioned over the transparent portion of the housing when the interface is removably coupled to the surface of the housing.

14. The wearable electronic device of claim 13, wherein the transparent region includes an aperture defined by the third portion of the interface.

15. The wearable electronic device of claim 13, wherein the transparent region includes a transparent material.

16. The wearable electronic device of claim 11, wherein the second portion is embedded within the third portion.

17. The wearable electronic device of claim 16, wherein:
a first surface of the first portion that contacts the first electrode is raised relative to a surface of the third portion that contacts the surface of the housing when the interface is removably coupled to the surface of the housing; and
a second surface of the first portion that contacts the second electrode is raised relative to the surface of the third portion.

18. The wearable electronic device of claim 17, wherein:
a surface of the first electrode is raised relative to the surface of the housing; and
a surface of the second electrode is raised relative to the surface of the housing.

19. The wearable electronic device of claim 15, wherein the housing has a ring shape to accommodate a finger of a user wearing the wearable electronic device.

20. The wearable electronic device of claim 10, wherein the second material includes at least one of an adhesive material or a magnetic material.
